(19) United States
(12) Patent Application Publication
(10) Pub. No.: US 2009/0149775 A1
(43) Pub. Date: Jun. 11, 2009

(54) Variable-Diameter Punch Biopsy

(76) Inventor: Harry Lander, New York, NY (US)

Correspondence Address: SILLS CUMMIS & GROSS P.C., ONE ROCKEFELLER PLAZA, IP DEPARTMENT - 25TH FLOOR, NEW YORK, NY, 10020, US

(21) Appl. No.: 11/952,662

(22) Filed: Dec. 7, 2007

(51) Int. Cl. A61B 10/02

(52) U.S. Cl. 600/567

(57) ABSTRACT

A punch biopsy device that includes a handle, a blade head, a blade, and a blade adjuster. The blade of the punch biopsy device is variable in diameter and is adjusted by the blade adjuster. That is, the blade adjuster adjusts the cylindrical opening of the blade so that the blade's diameter may be increased or decreased.

### BACKGROUND OF THE INVENTION

1. Field of the Invention

The present invention is related to a punch biopsy device, and more specifically, to a variable-diameter punch biopsy device.

2. Prior Art

In the medical field, particularly dermatology, many abnormalities found on the surface of a patient's skin, such as tumors or lesions, must be partially or fully removed before any tests or analyses are run on the sample. These abnormalities are usually excised by a simple surgical procedure. In the past, the surgical procedure was performed free-hand by a surgeon using a scalpel. That was, an incision was made around the skin abnormality to create an isolated piece of skin that was lifted by forceps to expose the underlying tissue. The tissue would then be cut away by a scalpel or scissors so the abnormality could be removed.

Over time, an easier surgical procedure was developed in which a skin-punch biopsy instrument was used for collecting specimens of tissue from the skin for analysis. The skin punch biopsy devices bore cylindrical channels into the skin by rotating a tubular cutting edge of the instrument into the tissue. A practitioner then lifted the circular hole formed by the instrument and used a scalpel to cut underneath the circular-shaped incision to free it from the subcutaneous tissues.

In practice though, each abnormality is unique in shape and size and each required sample also varies in size. Therefore, doctors need to purchase and have on hand many different size punches for removal of these abnormalities.

### SUMMARY OF THE INVENTION

The present invention provides a variable-diameter punch biopsy device that can be used to sample a wide range of different-sized abnormalities.

The punch biopsy device of the present invention comprises a handle, a blade head, a variable-diameter blade and a blade adjuster for adjusting the diameter of the blade. The handle preferably has an ergonomic design for easy handling and also a non-slip, gripping surface. The handle is made from a plastic, a rubber-coated metal or any type of known rigid material.

The blade head can be attached to the handle in a number of ways. For example, the blade may be (1) fixedly attached to the handle, (2) removably attached to the handle or (3) may be formed integral with the handle during manufacture.

The blade head houses a variable-diameter blade and a blade adjuster. The blade is held within the blade head by a holding means. The holding means may be at least two rods having compression springs located thereon or may be a piece of compression material such as silicone or rubber.

In a first embodiment, the blade itself may be a plurality of overlapping curved plates. The plurality of overlapping curved plates each has a cutting edge that cooperates to form a cylindrical blade opening. In this embodiment, when the diameter of the blade is decreased, a clamp compresses the plates so that the plates slide over each other. When the diameter of the blade increases, a clamp releases compression of the plates so that the plates slide away from each other.

In a second embodiment, the blade may be a band of metal. This band of metal has a first end and a second end with the first end overlapping the second end. The band of metal also has a cutting edge circumference that forms a cylindrical blade opening. In this embodiment, a captive screw assembly, such as a ring clamp, is used to tighten and loosen the band of metal thereby increasing and decreasing the diameter of the blade. This clamp is used in conjunction with a compression material which assists in opening the blade.

For transport purposes, the punch device may also include a blade cap so that the cutting edge may be protected and sterile before use.

### BRIEF DESCRIPTION OF THE DRAWINGS

These and other features, aspects, and advantages of the apparatus and methods of the present invention will become better understood with regard to the following description and accompanying drawings where:

FIG. 1 is a perspective view of the first embodiment of the invention;

FIG. 2 is a top view of the blade according to the first embodiment of the invention;

FIG. 3 is a perspective view of a single curved plate of the blade according to the first embodiment of the invention;

FIG. 4 is a side view of a rod according to the first embodiment of the invention;

FIG. 5 is top cross-sectional view of blade head according to the first embodiment of the invention;

FIG. 6 is a perspective view of the second embodiment of the invention;

FIG. 7 is a top view of the blade according to the second embodiment of the invention;

FIG. 8 is a perspective view of the blade according to the second embodiment of the invention;

FIG. 9 is top cross-sectional view of blade head according to the second embodiment of the invention; and

FIG. 10 is a perspective view of the blade adjuster.

### DETAILED DESCRIPTION OF THE INVENTION

The present invention provides a variable-diameter punch biopsy device that can be used to sample a wide range of different-sized abnormalities.

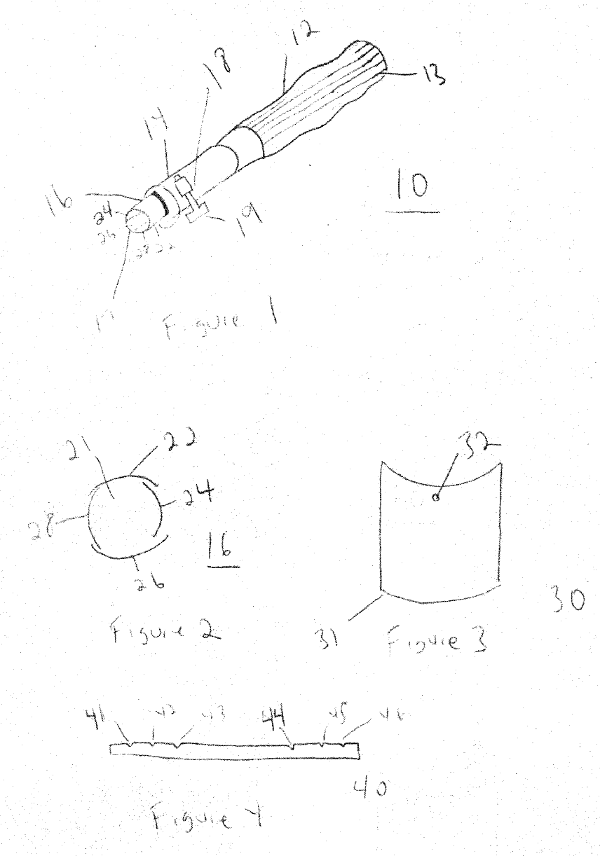

As shown in FIG. 1, the punch biopsy device 10 of the present invention includes a handle 12, a blade head 14, a blade 16 and a blade adjuster 18.

The handle 12 preferably has an ergonomic design for easy handling and also a non-slip, gripping surface 13. The handle 12 is made from a plastic, a rubber-coated metal or any other type of rigid material capable of being used as a handle.

The blade head 14 houses a variable-diameter blade 16 and a blade adjuster 18. The blade head 14 may be affixed to the handle 12 in a number of ways. For example, the blade head 14 may be a separate assembly that is adhered with an adhesive to the handle 12 so that the blade head 14 is permanently affixed to the handle 12. The blade head 14 may be removable from the handle 12 by a snap or screw thread assembly or the blade head 14 may be formed integral with the handle 12 during manufacture.

The blade adjuster 18 traverses the wall of the blade head 14. This allows a band section 46 (shown in FIG. 5) of the blade adjuster 18 to be located within the walls of the blade head 14 and a winged screw section 48 (shown in FIG. 5) of the blade adjuster 18 to be located on an outside wall of the blade head 14. This configuration allows a user to easily adjust the diameter of the blade's cutting edge 17.

As shown in FIG. 2, the blade 16 of the first embodiment is made of a plurality of overlapping curved plates 22, 24 26, 28. In this embodiment, four plates are used but three or more plates are contemplated. Each of these curved plates 22, 24 26, 28 cooperate to form a cylindrical blade opening 21. At the transition points between the curved plates 22, 24, 26 28 the plates lay completely flat against each other so that when the blade is inserted into the skin of a patient it makes a seamless circular hole in the skin.

The blade opening 21 is variable between an approximate minimum diameter of 0.25 cm and an approximate maximum diameter of 10 cm. The depth of the blade is about 1-4 cm. In the preferred embodiment the depth of the blade is not variable but a variable depth assembly is considered in the present invention.

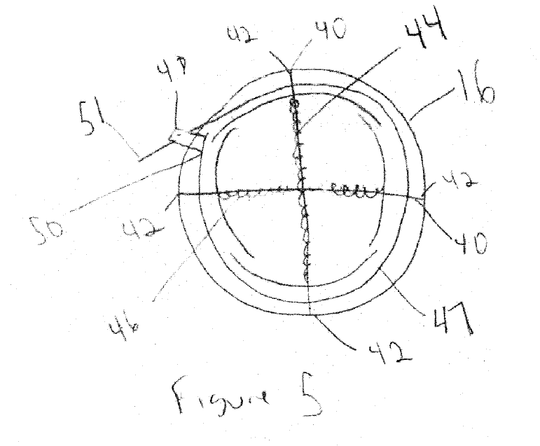

FIG. 3 shows an individual curved plate 30. This curved plate has a cutting edge 31 and an aperture 32. The aperture 32 is located on a top, center portion of the curved plate 30 and is used for holding the plate 30 within the blade head 14, as will be discussed in more detail below.

FIG. 4 shows a rod 40 having notches 41-46 located at specific intervals on the rod. The rods are also used for holding the plate 30 within the blade head 14 which is also discussed in more detail below.

FIG. 5 shows a cross sectional view of the blade head 14 with the blade 16 and blade adjuster 18. As shown in FIG. 5, in order to hold the blades within the blade head 14, the rods 40 traverse the interior of the blade head 14 and are anchored into the wall of the blade head 14 at points 42. These rods are also inserted into apertures 32 on the plates 22, 24 26, 28. The rods and apertures work in conjunction so that the blades are firmly seated within the blade head 14 and allow the blades to slide along the rods so that the circumference of the blade head 14 may be adjusted.

The notches 41-46 located on the rods can be used as length markers for the variable diameter of blade openings 21 because as the plates 22, 24, 26, 28 are moved vertically along the rod 40 the apertures 32 will slide into the notches 41-46 and the plates 22, 24, 26, 28 will frictionally lock into place within each notch 41-46. Each time a notch is encountered the blade will have been adjusted by a certain diameter. For example, the first set of notches may delineate that the blade opening is 5 cm and when the second set of notches is encountered the blade opening may 4 cm.

Also shown in FIG. 5 are compression springs 44, 46. These compression springs 44, 46 are located on the rods 40 on the interior side of the blade openings. These compression springs 44, 46 exert outward pressure to the blade opening 21 when the diameter of the blade opening is being increased.

As can be seen in FIG. 5, the blade is surrounded by a portion of the blade adjuster 18. The blade adjuster 18 exerts an inward cylindrical force around the circumference of the blade 16.

The blade adjuster 18 may be a screw clamp that includes a band 47, which is usually thin flexible metal, into which a screw thread pattern (not shown) has been cut. One end 50 of the band contains a captive screw assembly 48 while the other end 51 is loose and is fed into a narrow space between the band 47 and the captive screw assembly 48. The captive screw assembly 48 includes a winged screw 19 that when turned clockwise, pulls the threads of the band 47, causing the band 47 to tighten around the blade 16 and when turned counter-clockwise loosens the band 47 around the blade 66.

The compression springs 44, 46 and the blade adjuster 18 work in conjunction to increase and decrease the diameter of the blade opening. Therefore, in use, when a user wants to decrease the diameter of the blade 16. The user turns the winged screw 19 located on the outer surface of the blade head 14 which in turn causes the circumference of the band to decrease. This pressure causes the plates 22, 24, 26, 28 of the blades 16 to slide over each other and compress the compression springs 44, 46.

If a user wants to increase the diameter the blade 16, a user turns the blade adjuster 18 in the opposite direction which causes the band 47 to open. The compressed compression springs 44, 46 exert an outward pressure on the plates 22, 24, 26, 28 so that plates 22, 24, 26, 28 slide away from each other thereby increasing the diameter of the blade 16. The notches 41-46, as discussed above, may provide the user with a marker indicating the size of the opening 21.

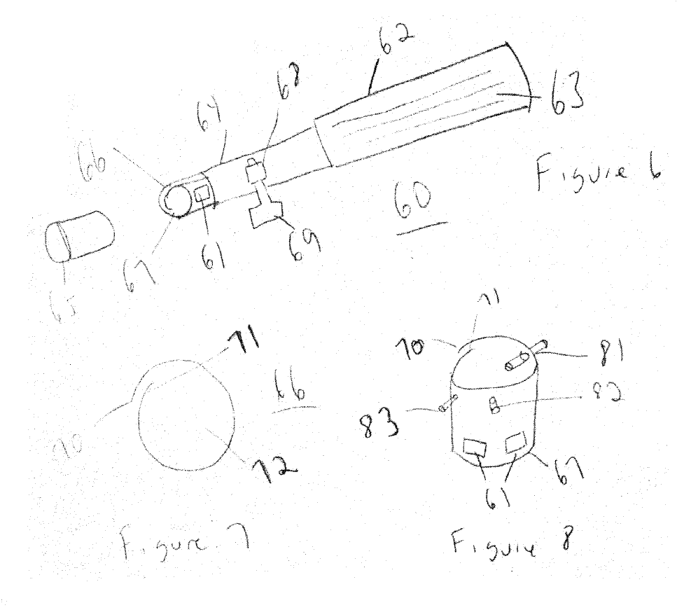

In a second embodiment, as shown in FIG. 6, the punch biopsy device 60 of the present invention includes a handle 62, a blade head 64, a blade 66 and a blade adjuster 68.

The handle 62 may have an ergonomic design for easy handling and also has a non-slip, gripping surface 63. The handle 62 is made from a plastic, a rubber-coated metal or any type of rigid material.

The blade head 64 houses a variable-diameter blade 66 and a blade adjuster 68. The blade head 64 may be affixed to the handle 62 in a number of ways. For example, the blade head 64 may be a separate assembly that is adhered with an adhesive to the handle 62 so that the blade head 64 is permanently affixed to the handle 62. The blade head 64 may be removable from the handle 62 by a snap or screw thread assembly or the blade head 64 may be formed integral with the handle 62 during manufacture.

The blade adjuster 68 traverses the wall of the blade head 64. This allows the band section 91 (shown in FIG. 9) of the blade adjuster 68 to be located within the walls of the blade head 64 and the winged screw assembly 69 to be located on the outside of the blade head 64. This configuration allows the user to easily adjust the diameter of the blade's cutting edge 67.

As shown in FIG. 7, the blade 66 is a band of metal having a leading end 70 and a trailing end 71. The leading end 70 and the trailing end 71 overlap to form a cylindrical opening 72. The cylindrical opening 72 varies between approximately 10 cm at its maximum and approximately 0.25 cm at its minimum. At the transition point between the leading edge 70 and trailing edge 71, these edges 70, 71 lay completely flat against each other so that when the blade 66 is inserted into the skin of a patient it makes a seamless circular hole in the skin.

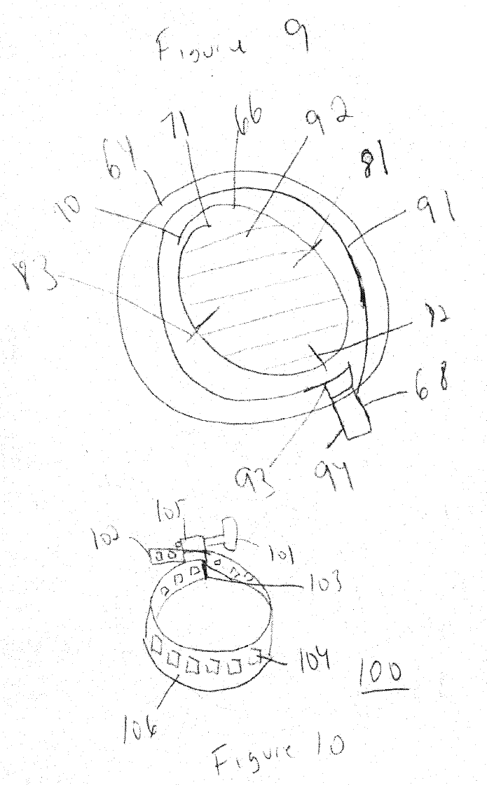

In FIG. 8, it is shown that the blade 66 may have an aperture 61 located slightly above the cutting edge 67 so that a practitioner can visually see that a particular abnormality is within the circumference of the blade opening 72 before an incision is made ensuring that the practitioner aligned the blade 66 opening fully around the abnormality.

The blade 66 is held within the blade head 64 by a compression material 92 such as a silicone or rubber material. The compression material 92 is first formed and anchored to the interior walls of the blade head 64. The top portion of the blade 66 is then inserted into the material 92 thereby holding the blade 66 in place.

In another embodiment, to further ensure the blade 66 is firmly seated in the compression material 92, the compression material 92 may be formed around the blade 66 before both are anchored within the blade head 64. In this embodiment, the blade 66 may include pins 81-83 located on the top portion of the blade 66. These pins 81-83 give the blade 66 added surface area so that the compression material 92 and the blade 66 are adhered to each other without the possibility of the blade 66 falling out of the compression material 92. The pins 81-83 may be either inserted into apertures located on the blade 66 or they may be welded or soldered to the blade 66. In either case, the pins 81-83 add support when the blade 66 and the compression material 92 are married together.

The compression material 92 has the ability to be compressed and hold the force of a compression until the pressure is released. That is, in a rest state the material 92 will not exert any outward force upon the blade 66 but when the blade's diameter 72 is decreased the compression material 92 retains the inward force. Therefore, when the pressure is released the compression material 92 returns to its rest state and to do so will exert an outward force on the blade 66 thereby increasing the diameter of the blade opening 72.

A blade adjuster 68 similar to that of the first embodiment surrounds the outer circumference of the blade 66. Specifically, the blade adjuster 68 may be a screw clamp that consists of a band 91 (usually thin flexible metal) into which a screw thread pattern has been cut. When the blade adjuster 68 is tightened, the screw clamp assembly 94 asserts a cylindrical force upon the blade 66 making the leading section 70 slide further along the band of metal thereby decreasing the size of the blade 66 and in reverse the loosening of the adjuster 68 allows the pressure of the band 91 to be removed. This movement in conjunction with the compressed material 92 allows the blade 66 to open. When opening, the leading edge 70 slides towards the trailing edge 71 increasing the diameter of the blade edge 67.

In order to safely transport the punch devices the blade has a blade cap 65 for protecting the cutting edge of the device and keeps the blade sterile. The blade cap 65 may be a plastic or rubber material. The device may also include a means for cutting the sample away from the tissue of the skin without the use of a scalpel.

FIG. 10 shows the preferred embodiment for the blade adjuster 100 of the present invention. The blade adjuster 100 includes a band 106 made from a thin flexible metal into which a screw thread pattern 104 has been cut and a captive screw assembly 105. The leading end 103 of the band is attached to the captive screw assembly 105 while the trailing end 102 is loose and is fed into a narrow space between the leading end 103 of the band and the captive screw assembly 105. The captive screw assembly 205 includes a winged screw 101 that when turned clockwise, pulls the threads of the band 106, causing the band 106 to tighten around the blade 16 or 66 and when turned counter-clockwise loosens the band 106 around the blade 16 or 66.

Although the punch biopsy device of the present invention has been described in detail and with particularity, it will be appreciated by those skilled in this art that changes and modifications can be made therein without departing from the scope and spirit of the invention.

What is claimed:

1. A punch biopsy device comprising:
a handle;
a blade head;
a blade, the blade having a diameter, the diameter being variable; and
a blade adjuster for adjusting the diameter of the blade.

2. The punch biopsy device as claimed in claim 1 wherein the handle has an ergonomic design for easy handling and has a non-slip, gripping surface.

3. The punch biopsy device as claimed in claim 1 wherein the blade head is fixedly attached to the handle.

4. The punch biopsy device as claimed in claim 1 wherein the blade head is removably attached to the handle.

5. The punch biopsy device as claimed in claim 1 wherein the blade head and the handle are formed integral with each other.

6. The punch biopsy device as claimed in claim 1 wherein the blade head houses the blade and the blade adjuster.

7. The punch biopsy device as claimed in claim 6 wherein the blade is held within the blade head by a holding means.

8. The punch biopsy device as claimed in claim 7 wherein the holding means is at least two rods that traverse and are anchored into interior walls of the blade head.

9. The punch biopsy device as claimed in claim 8 wherein the rods have notches for locking the blade in place.

10. The punch biopsy device as claimed in claim 9 wherein the rods include compression springs.

11. The punch biopsy device as claimed in claim 7 wherein the holding means is a compression material.

12. The punch biopsy device as claimed in claim 10 wherein the blade is a plurality of overlapping curved plates that form an opening.

13. The punch biopsy device as claimed in claim 12 wherein the plurality of overlapping curved plates each have a cutting edge that cooperate to form a cylindrical blade opening.

14. The punch biopsy device as claimed in claim 13 wherein, when the diameter of the blade is decreased, a clamp compresses the plates so that the plates slide over each other, and, when the diameter of the blade increased, a clamp releases compression exerted on the compression springs so that the plates slide away from each other.

15. The punch biopsy device as claimed in claim 11 wherein the blade is a band of metal, the band of metal having a first end and a second end, the first end overlapping the second end forming an opening.

16. The punch biopsy device as claimed in claim 15 wherein the band of metal has a cutting edge circumference that forms a cylindrical blade opening.

17. The punch biopsy device as claimed in claim 16 wherein, when the diameter of the blade is decreased, a clamp compresses the band of metal so that the first end slides over the second end, and, when the diameter of the blade increased, a clamp releases compression of the compression material so that the first end of the band of metal slides away from the second end.

18. The punch biopsy device as claimed in claim 14 wherein the clamp is a captive screw assembly.

19. The punch biopsy device as claimed in claim 17 wherein the clamp is a ring clamp.

20. The punch biopsy device as claimed in claim 13 further comprising:
a blade cap for protecting the cutting edge of the device.
